(19) United States
(12) Patent Application Publication
(10) Pub. No.: US 2025/0235099 A1
(43) Pub. Date: Jul. 24, 2025

(54) DETECTING AN INTRAOCULAR LENS IN AN IMAGE OF AN EYE

(71) Applicant: Alcon Inc., Fribourg (CH)

(72) Inventor: Martin Gründig, Rangsdorf (DE)

(21) Appl. No.: 19/028,559

(22) Filed: Jan. 17, 2025

Related U.S. Application Data
(60) Provisional application No. 63/623,101, filed on Jan. 19, 2024.

(51) Int. Cl. A61B 3/14; G06T 7/00

(52) U.S. Cl. CPC A61B 3/145; G06T 7/0012; G06T 2207/10101; G06T 2207/30041

(57) ABSTRACT

In certain embodiments, a system for detecting an intraocular lens (IOL) in an image of an eye includes an imaging system and a computer system. The imaging system generates the image of the eye. The eye has multiple structures, including the IOL. The computer system receives the image of the eye from the imaging system and detects one or more Purkinje image(s) in the image of the eye. A Purkinje image comprises a reflection from a structure of the eye. The computer system uses the Purkinje image(s) to detect one or more surface(s) of the IOL and determines a location of the IOL according to the surface(s) of the IOL.

### RELATED APPLICATION

This application is related to U.S. Provisional Patent Application No. 63/623,101, titled, “PURKINJE GUIDED SEGMENTATION”, filed 19 Jan. 2024, which is incorporated by reference herein in its entirety.

### TECHNICAL FIELD

The present disclosure relates generally to eye imaging, and more particularly to detecting an intraocular lens in an image of an eye.

### BACKGROUND

For intraocular lens (IOL) surgery, a doctor may use pre-operation (“pre-op”) patient data, such as a pre-op image of the patient's eye, to select an IOL design. The doctor may then implant the IOL into the eye during the surgery. After healing from the surgery, post-operation (“post-op”) patient data, such a post-op image of the pseudophakic eye, may be obtained. The pre-op and post-op patient data can be analyzed to improve the results of future surgeries.

### BRIEF SUMMARY

In certain embodiments, a system for detecting an intraocular lens (IOL) in an image of an eye includes an imaging system and a computer system. The imaging system generates the image of the eye. The eye has multiple structures, including the IOL. The computer system receives the image of the eye from the imaging system and detects one or more Purkinje image(s) in the image of the eye. A Purkinje image comprises a reflection from a structure of the eye. The computer system uses the Purkinje image(s) to detect one or more surface(s) of the IOL and determines a location of the IOL according to the surface(s) of the IOL.

Certain embodiments of the invention may include one, two or more, any suitable combination of, or all of the following.
The imaging system comprises an optical coherence tomography (OCT) imaging system that generates the image of the eye as an OCT image.
The computer system detects the Purkinje image(s) in the image of the eye by: detecting an iris and a pupil in the image of the eye; creating one or more enface image(s) according to the iris and the pupil; and detecting the Purkinje image(s) in at least one of the enface image(s).
The computer system detects the Purkinje image(s) in the image of the eye by: detecting an iris plane in the image of the eye; creating an iris enface image using the iris plane; detecting a pupil in the iris enface image; creating a pupil enface image posterior to the iris plane; and detecting the Purkinje image(s) in the pupil enface image.
The computer system uses the Purkinje image(s) to detect the surface(s) of the IOL by: defining a search volume according to a Purkinje image of the Purkinje image(s); and searching the search volume for a surface of the surface(s) of the IOL.
The computer system uses the Purkinje image(s) to detect the surface(s) of the IOL by: determining an enface location of a point associated with a Purkinje image, where the point corresponds to a Purkinje point reflecting light that yields the Purkinje image; and detecting a surface of the IOL according to the enface location of the point associated with the Purkinje image.
The computer system uses the Purkinje image(s) to detect the surface(s) of the IOL by: determining a depth location of a point associated with a Purkinje image, the point corresponding to a Purkinje point reflecting light that yields the Purkinje image; and detecting a surface of the IOL according to the depth location of the point associated with the Purkinje image.
The computer system uses the Purkinje image(s) to detect the surface(s) of the IOL by determining a location of a point associated with a Purkinje image, the point corresponding to a Purkinje point constraining a surface of the IOL to include the location of the point.
The computer system uses the Purkinje image(s) to detect the surface(s) of the IOL by detecting a posterior surface of the IOL and searching a volume that is anterior to the posterior surface for an anterior surface of the IOL.
The computer system uses the Purkinje image(s) to detect the surface(s) of the IOL by detecting a posterior surface of the IOL and detecting an anterior surface of the IOL according to the posterior surface and a shape of the IOL.
The computer system detects the Purkinje image(s) in the image of the eye by detecting a fourth Purkinje (P4) image, where the P4 image is reflected from a posterior surface of the IOL. The computer system uses the Purkinje image(s) to detect the surface(s) of the IOL by using the P4 image to detect the posterior surface of the IOL.
The computer system detects the Purkinje image(s) in the image of the eye by detecting a fourth Purkinje (P4) image, where the P4 image is reflected from a posterior surface of the IOL. The computer system uses the Purkinje image(s) to detect the surface(s) of the IOL by: determining a location of a P4 point that yields the P4 image; defining a posterior search volume according to the location of the P4 point; and searching the posterior search volume for the posterior surface of the IOL.
The computer system detects the Purkinje image(s) in the image of the eye by detecting a fourth Purkinje (P4) image, where the P4 image is reflected from a posterior surface of the IOL. The computer system uses the Purkinje image(s) to detect the surface(s) of the IOL by: determining a location of a P4 point that yields the P4 image; and constraining a surface of the IOL to include the location of the P4 point.
The computer system detects the Purkinje image(s) in the image of the eye by detecting a fourth Purkinje (P4) image, where the P4 image is reflected from a posterior surface of the IOL. The computer system uses the Purkinje image(s) to detect the surface(s) of the IOL by: determining a depth of a maximum intensity of the P4 image; and detecting the posterior surface using the depth of the maximum intensity of the P4 image.
The computer system detects the Purkinje image(s) in the image of the eye by detecting a third Purkinje (P3) image, where the P3 image is reflected from an anterior surface of the IOL. The computer system uses the Purkinje image(s) to detect the surface(s) of the IOL by using the P3 image to detect the anterior surface of the IOL.
The computer system detects the Purkinje image(s) in the image of the eye by detecting a third Purkinje (P3) image, where the P3 image is reflected from an anterior surface of the IOL. The computer system uses the Purkinje image(s) to detect the surface(s) of the IOL by: determining a location of a P3 point that yields the P3 image; defining an anterior search volume according to the location of the P3 point; and search the anterior search volume for the anterior surface of the IOL.
The computer system detects the Purkinje image(s) in the image of the eye by detecting a third Purkinje (P3) image, where the P3 image is reflected from an anterior surface of the IOL. The computer system uses the Purkinje image(s) to detect the surface(s) of the IOL by: determining a location of a P3 point that yields the P3 image; and constraining a of the IOL to include the location of the P3 point.
The computer system detects the Purkinje image(s) in the image of the eye by detecting a third Purkinje (P3) image, where the P3 image is reflected from an anterior surface of the IOL. The computer system uses the Purkinje image(s) to detect the surface(s) of the IOL by: determining a depth of a maximum intensity of the P3 image; and detecting the anterior surface using the depth of the maximum intensity of the P3 image.
The computer system displays the image of the eye, receives a user selection of a search volume, and searches the search volume for the surface(s) of the IOL.
The computer system displays the image of the eye, displays a search volume on the image of the eye, and searches the search volume for the surface(s) of the IOL.

### BRIEF DESCRIPTION OF THE DRAWINGS

FIG. 1 illustrates an example of a system that can generate an image of an eye and detect an IOL in the image, according to certain embodiments;

FIG. 2 illustrates an example of an optical coherence tomography (OCT) system that can generate images of the interior of an eye, according to certain embodiments;

FIG. 3 illustrates an example of a display that may be used to display images of an eye, according to certain embodiments;

FIG. 4 illustrates an example of a method of detecting an IOL in an image of an eye, according to certain embodiments; and

FIGS. 5 and 6A to 6I illustrate an example of a method of detecting an IOL in an image of an eye, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Pre-op and post-op images of the eye can be analyzed to improve the results of IOL surgeries. Optical coherence tomography (OCT) may be used to obtain images of the eye, e.g., the pseudophakic eye after surgery. However, an IOL surface does not reflect light in a manner readily detectable by an OCT sensor. An IOL surface is smooth and highly reflective, so tends to yield less diffuse back-reflections, especially if the surface is not facing the OCT sensor. This problem is exacerbated if the noise is high, such as noise from signals from the capsular bag or floaters in the vitreous humour. Accordingly, the IOL surfaces typically do not show up well in OCT images, which makes segmenting the image to identify the IOL difficult. In known approaches to addressing poor segmentation, the operator of the device visually inspects the segmentation results and may correct the segmentation. However, manual inspection can be unreliable and inconsistent, so cases that require manual inspection should be minimized.

Certain embodiments described herein utilize specific points of the IOL surfaces that yield strong specular reflections, i.e., Purkinje images, readily detectable by an OCT sensor. For example, the reflection from the point posterior surface of the IOL yields the P4 Purkinje image, which typically shows up in an OCT image and indicates where the posterior surface may be located. Accordingly, a volume around the point may be searched to segment the posterior surface. In a similar manner, the P3 Purkinje image may be used to search for the anterior surface of the IOL. Given the posterior surface and the anterior surface of the IOL, the location of the IOL may be determined.

Certain embodiments may provide advantages. For example, embodiments may identify IOL surfaces according to Purkinje reflections, which may reduce segmentation errors. As another example, embodiments may automatically identify IOL surfaces, which may reduce the need for manual verification.

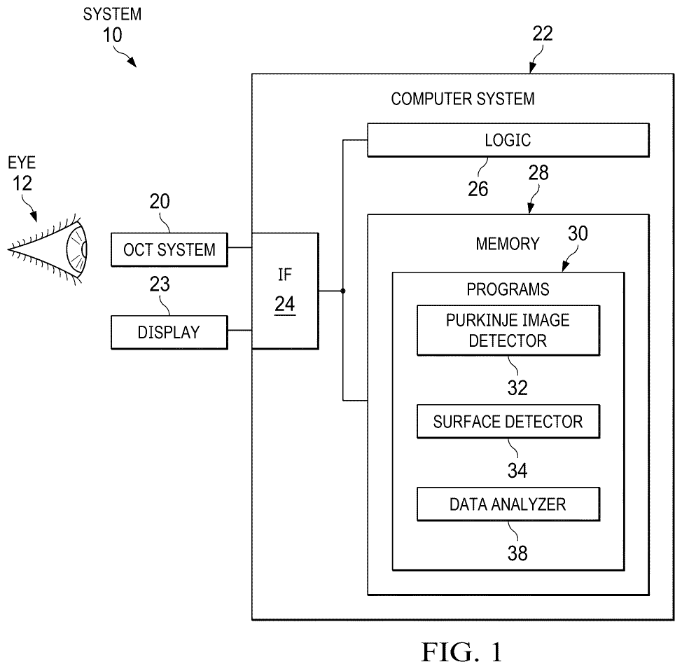

FIG. 1 illustrates an example of a system 10 that can generate an image of an eye 12 and detect an IOL in the image, according to certain embodiments. In general, the surfaces of an IOL do not reflect light well, except for at regions that yield Purkinje images. To search for the IOL, the system 10 uses Purkinje images to target regions of the eye where the IOL is more likely to be located.

In the example, the system 10 includes an optical coherence tomography (OCT) system 20, a computer system 22, and a display 23, coupled as shown. The computer system 22 includes an interface (IF) 24, logic 26, and a memory 28, coupled as shown. The memory 28 stores programs 30, such as a Purkinje image detector 32, a surface detector 34, and a data analyzer 38.

In an example of operation, an imaging system (e.g., the OCT system 20) generates an image of eye 12 that has an intraocular lens (IOL). The computer system 22 receives the image of the eye 12, which has structures, including the IOL. The Purkinje image detector 32 detects one or more Purkinje images in the image of the eye, where a Purkinje image is a reflection from a structure of eye 12. The surface detector 34 uses the one or more Purkinje images to detect one or more surfaces of the IOL to determine a location of the IOL in the eye 12. The data analyzer 38 may use the location of the IOL to analyze data pertaining to the effectiveness of IOLs.

For ease of explanation, certain features of the imaging system may be used to define an example coordinate system used herein. For example, the imaging system may have an optical axis that defines the z-axis, which in turn defines an xy-plane of the coordinate system. A z-location indicates the location relative to the z-axis, and an xy-location indicates a location relative to the xy-plane. The eye may be generally aligned such that the z-axis is generally aligned with an eye axis (e.g., optical or pupillary axis).

OCT System. Turning to examples of the components of the system 10, the OCT system 20 is an example of any suitable imaging system that generates an image of eye 12, e.g., the OCT system 20 generates an OCT image of the eye 12. In general, the imaging system directs light towards eye 12, and surfaces (e.g., the anterior surface and/or the posterior surface) of the structures of the eye 12 (e.g., the cornea, the iris, the lens, an IOL, and/or the retina) reflect the light. The imaging system detects the reflected light to generate an image of the eye 12. The eye image may show the surfaces of the structures, but sometimes the surfaces are not distinct, so the system 10 analyzes the image to detect the surfaces.

Purkinje Image Detector. A Purkinje image is a reflection from a structure of the eye 12. At least four Purkinje images are usually visible in an eye with the natural crystalline lens. The first Purkinje (P1) image is the reflection from the anterior surface of the cornea. The second Purkinje (P2) image is the reflection from the posterior surface of the cornea. The third Purkinje (P3) image is the reflection from the anterior surface of the lens (natural or IOL). The fourth Purkinje (P4) image is the reflection from the posterior surface of the lens. Unlike the other images, the P4 image is an inverted image.

In a pseudophakic eye, the natural crystalline lens has been replaced by an IOL, which typically does not show up well in an OCT image. In general, only the points (described herein as “Purkinje points”) of the IOL surfaces that directly face towards the OCT sensor yield strong specular reflections, i.e., Purkinje images, readily detectable by an OCT sensor. The reflection from the point on the posterior surface of the IOL (the “P4 point”) yields the P4 Purkinje image. The reflection from the point on the anterior surface of the IOL (the “P3 point”) yields the P3 Purkinje image. The P4 point is farther away in the z-direction from the sensor than the P3 point is, so the P4 point typically yields a stronger reflection and a brighter Purkinje image.

The Purkinje image detector 32 detects one or more Purkinje images in the image of the eye and may detect a Purkinje image in any suitable manner. In certain embodiments, the Purkinje image detector 32 identifies structures of the eye (e.g., the iris, the iris plane, and/or the pupil) in the eye image. The Purkinje image detector 32 creates one or more enface images relative to the structures and then searches an enface image for a Purkinje image. For example, the Purkinje image detector 32 detects the iris plane in the image of the eye and creates an iris enface image with respect to the iris plane, e.g., creates an iris enface image that is at an xy-plane at, near, or intersects the iris plane or that is at a plane parallel to or at the iris plane. Then, the Purkinje image detector 32 detects the pupil in the iris enface image and creates a pupil enface image posterior to the pupil area of the iris plane. The Purkinje image detector 32 detects a Purkinje image in the pupil enface image.

Surface Detector. The surface detector 34 detects the surfaces of structures (e.g., an IOL) in an eye using Purkinje images and may detect surfaces in any suitable manner. In certain embodiments, the surface detector 34 may determine the Purkinje point of a surface that reflects light that yields a Purkinje image. The surface detector 34 may determine the location (e.g., the xy-coordinates (enface), the z-coordinate (depth), and/or the xyz-coordinates) of the Purkinje point (as described in more detail herein) and then use the location to detect a surface. For example, the P4 Purkinje point (of the posterior surface) may be used to detect the posterior surface, and the P3 Purkinje point (of the anterior surface) may be used to detect the anterior surface.

In certain embodiments, the surface detector 34 uses a Purkinje point to define a search volume in the image where one or more particular surfaces are likely to be located and then searches the volume for the surface(s). For example, the P4 Purkinje point may be used to define a search volume for the posterior surface, and the P3 Purkinje point may be used to define a search volume for the anterior surface. As another example, the P4 Purkinje point may be used to define a search volume for the anterior and the posterior surfaces, where the P4 Purkinje point is located at or near the posterior side of the search volume. Similarly, the P3 Purkinje point may be used to define a search volume for the anterior and the posterior surfaces, where the P3 Purkinje point is located at or near the anterior side of the search volume. The surface detector 34 may search the volume with the constraint that the Purkinje point should be a point on the surface.

A search volume may have any suitable size or shape, e.g., a size and/or shape that may be slightly larger (such as less than 5, 5 to 10, 10 to 20% larger) than the surface(s) to be located. Examples of search volumes include: (1) a polyhedron (e.g., a hexahedron); (2) a lens-shaped volume with a convex anterior spherical surface and a convex posterior spherical surface; (3) a lens-shaped volume slightly larger than the average IOL; and/or (3) a lens-shaped volume slightly larger than the specific IOL.

In certain embodiments, the surface detector 34 detects a surface (e.g., anterior or posterior) of a structure of the eye 12 according to a previously detected surface (e.g., posterior or anterior, respectively) of the eye 12. For example, the surface detector 34 detects the posterior surface of the IOL and searches a volume anterior to the posterior surface of the IOL for the anterior surface of the IOL, or vice versa. As another example, the surface detector 34 detects the posterior surface of the IOL and determines the anterior surface of the IOL according to the expected shape of the IOL, or vice versa.

Data Analyzer. The data analyzer 38 may analyze pre-op and post-op patient data to evaluate the efficacy of the IOL surgery. For example, the data can be analyzed to determine the effect of the IOL on changing the patient's vision. The efficacy is affected by the position (e.g., the location and orientation) of the IOL in the eye, which can be obtained from the surface detector 34.

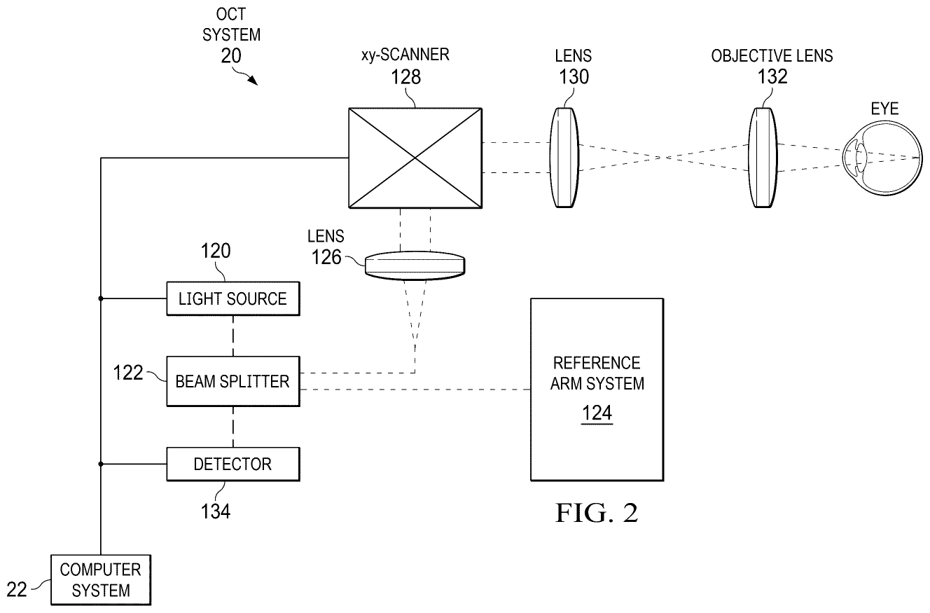

FIG. 2 illustrates an example of an optical coherence tomography (OCT) system 20 that can generate images of the interior of an eye, according to certain embodiments. In the illustrated example, the OCT system 20 includes a light source 120, a beamsplitter 122, a reference arm system 124, a lens 126, an xy-scanner 128, a lens 130, an objective lens 132, a detector 134, and a computer system 22, coupled as shown. The OCT system 20 may be used in the system 10 of FIG. 1.

As an overview of operation, the light source 120 provides a light beam. The beamsplitter 122 splits the light beam into a sample beam and a reference beam. Optical elements (e.g., the lens 126, the xy-scanner 128, the lens 130, the objective lens 132) direct the sample beam towards an eye, which reflects the light to yield a reflected sample beam. The reference arm system 124 directs the reference beam along a reference arm to yield a reflected reference beam. The surface detector 34 detects the reflected sample beam and the reflected reference beam, and generates a detector signal in response to detecting the beams. The computer system 22 determines image information from the detector signal and generates an image of the sample from the image information for the sample path ranges.

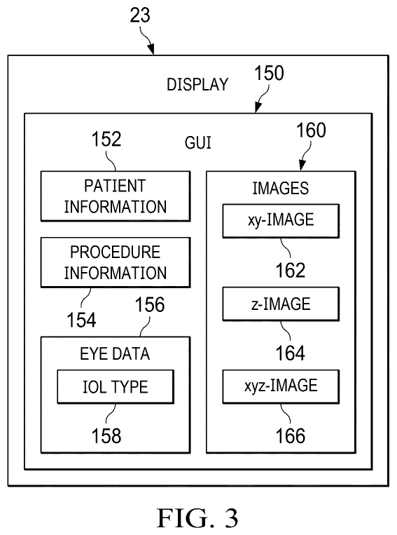

FIG. 3 illustrates an example of a display 23 that may display images of the eye, according to certain embodiments. The display 23 may be used in the system 10 of FIG. 1. In the example, the display 23 displays a graphical user interface (GUI) 150. The GUI 150 includes graphical sections 152, 154, 156, and 160 that can display and/or receive information of any suitable format, e.g., characters (such as text, numbers, and symbols) or images. The graphical sections include a patient information section 152, a procedure information section 154, an eye data section 156, and an images section 160.

In the example, the patient information section 152 includes information that identifies the patient. The procedure information section 154 indicates the type of surgery to be performed. The eye data section 156 includes an IOL type field 158 that can display and/or receive the type of IOL in the eye 12. The type of IOL may indicate the shape of the IOL, which may be used to determine the position of the IOL in an image. The images section 160 may include one or more of any suitable image. In the example, the images section 160 includes an xy-image 162, a z-image 164, and/or an xyz-image 166 of the images of the eye. An xy-image 162 is an enface image taken at an xy-plane at any suitable z-location. A z-image 164 is a depth image taken parallel to the z-axis. An xyz-image 166 is a three-dimensional (3D) image. Other examples of images are described herein.

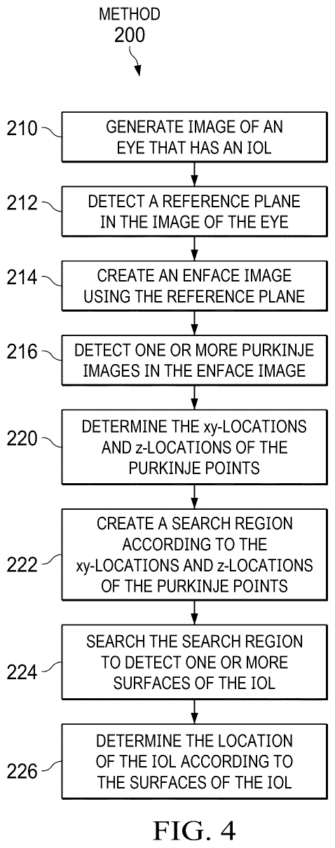

FIG. 4 illustrates an example of a method 200 for detecting an IOL in an image of an eye, according to certain embodiments. One or more operations of the method may be performed by embodiments disclosed herein, e.g., by a computer such as the computer system 22 of FIG. 1.

The method starts at block 210, where an imaging system generates an image of an eye that has an implanted IOL. A computer detects a reference plane in the image of the eye at block 212. The reference plane may be any suitable plane that indicates the location of an eye structure. For example, the reference plane may be the iris plane defined by the iris of the eye. At block 214, the computer creates an enface image using the reference plane. For example, the computer may create an iris enface image that is substantially parallel to or at the iris plane, detect the pupil of the eye in the iris enface image, and create a pupil enface image that is posterior to and substantially parallel to the pupil of the iris plane.

The computer detects one or more Purkinje images in the enface image at block 216. For example, the computer may detect a Purkinje image in the pupil enface image. The computer determines the xy-locations and z-locations of the Purkinje points that yield the Purkinje images at block 220. For example, the computer may detect the xy-location of a Purkinje image in the pupil enface image and then determine at which z-location the Purkinje image has the maximum intensity to detect the z-location of the Purkinje image.

At block 222, the computer creates a search region according to the xy-locations and z-locations of the Purkinje points. For example, the computer may create a search volume that includes the xy-locations and z-locations. The computer searches the search region to detect one or more surfaces of the IOL at block 224. For example, the computer may search the search volume with the xy-location and z-location of a P4 Purkinje point as a forced point to locate a posterior surface of the IOL. The computer may then use the posterior surface of the IOL to detect the anterior surface of the IOL. At block 226, the computer determines the location of the IOL according to the locations of one or more surfaces of the IOL.

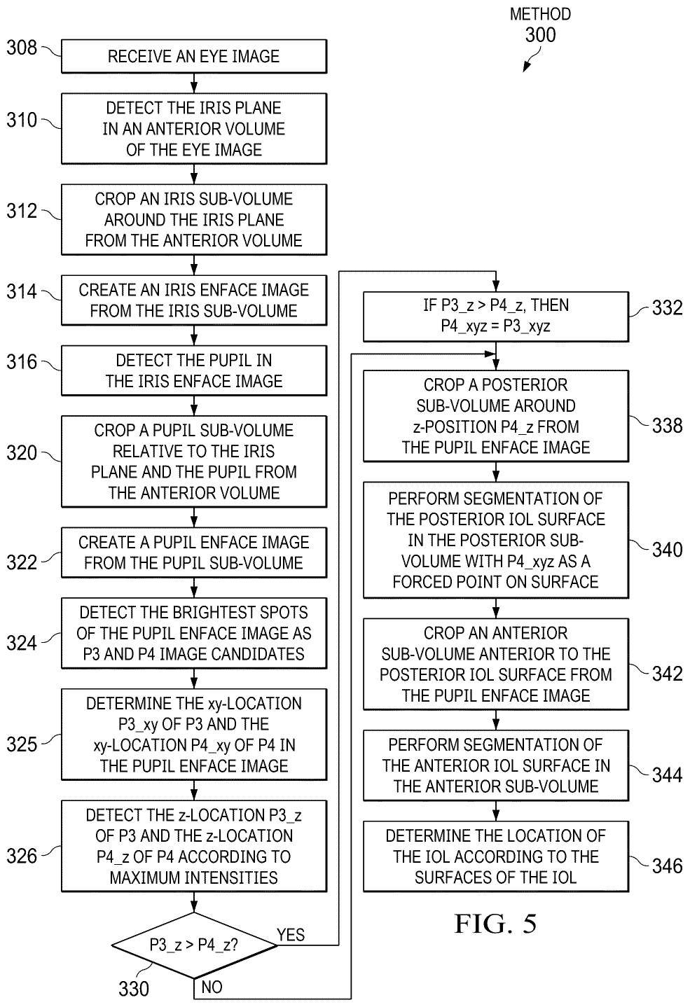

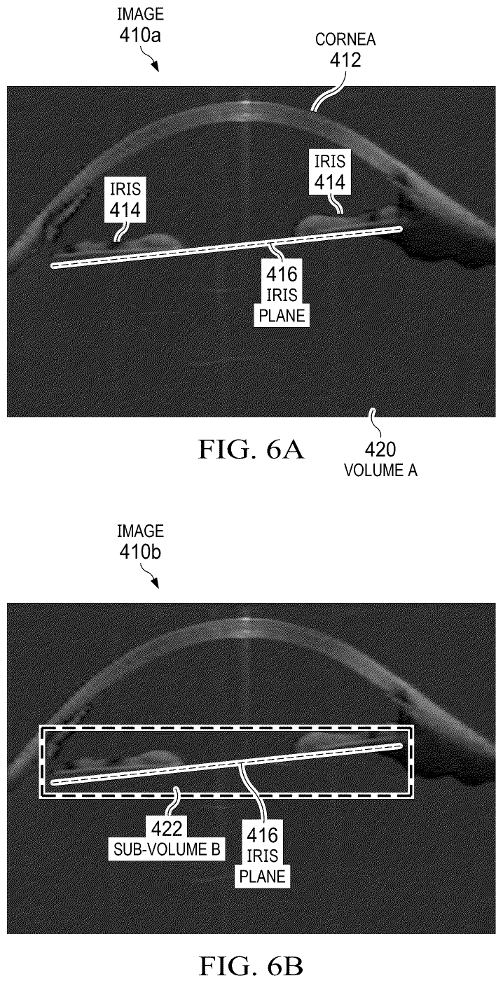

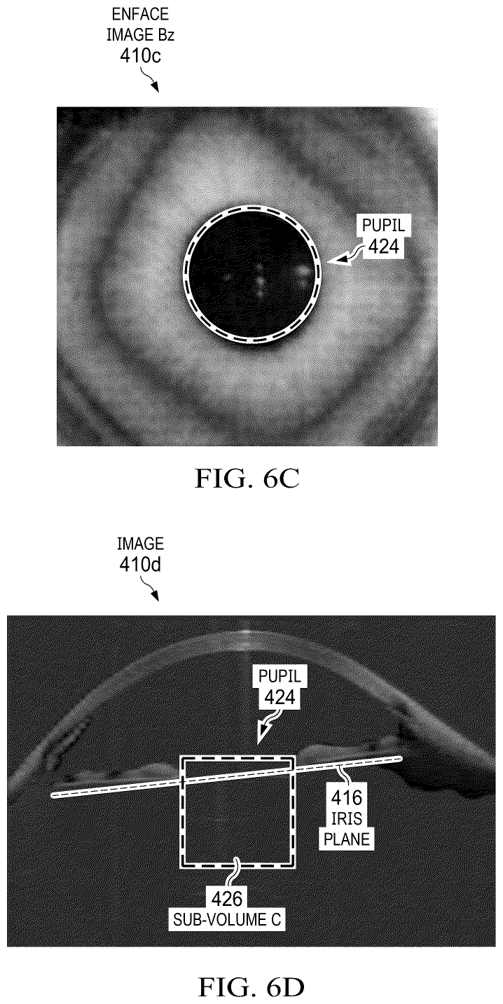

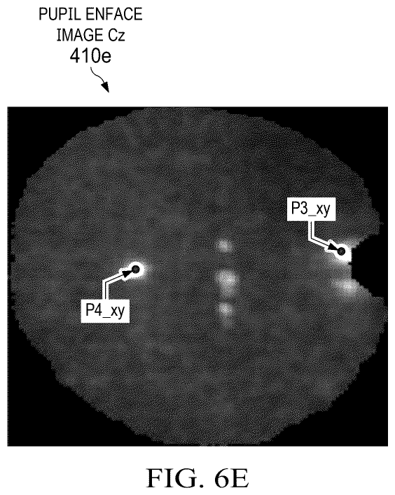

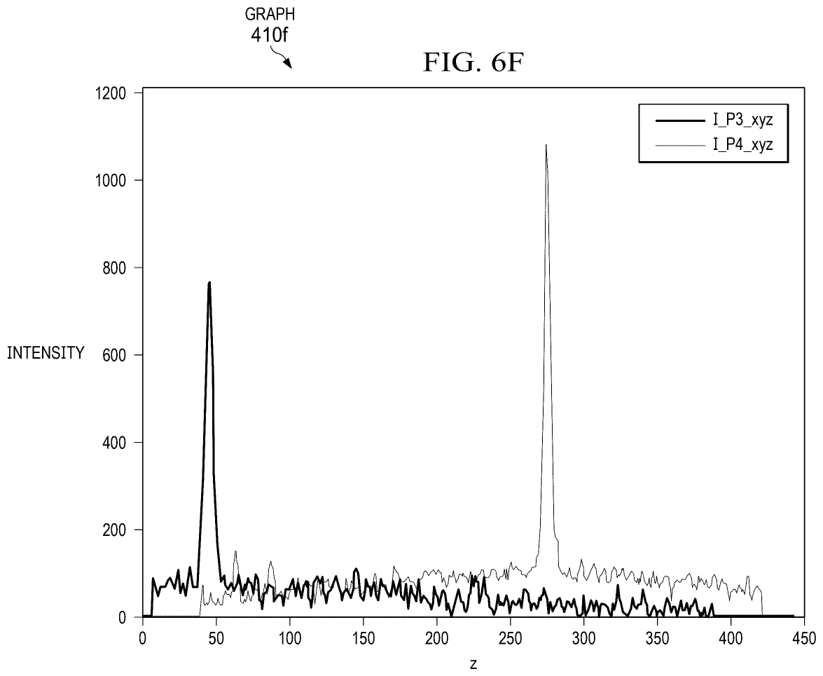

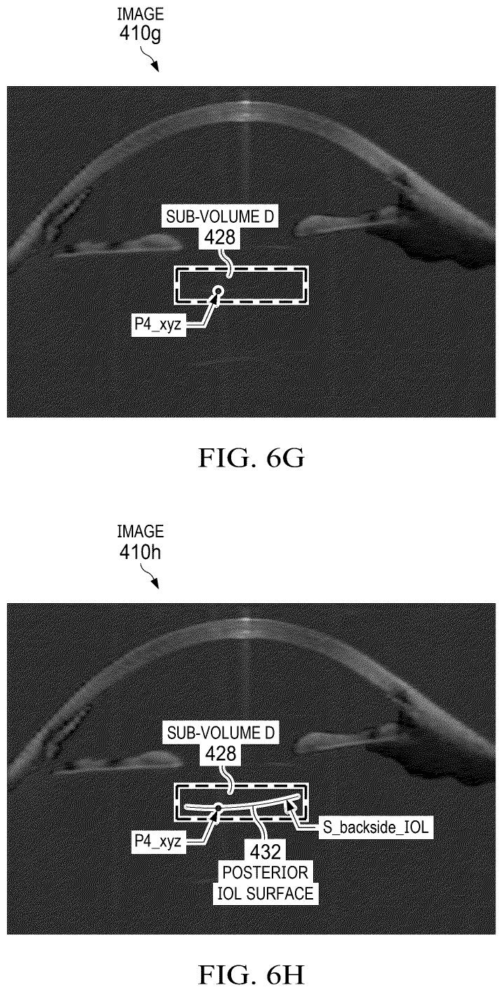

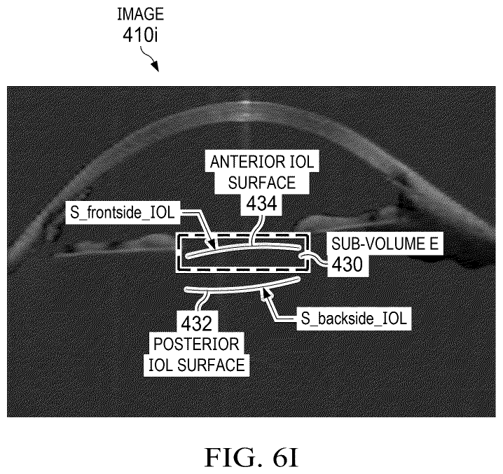

FIGS. 5 and 6A to 6I illustrate an example of a method 300 for detecting an IOL in an image of an eye, according to certain embodiments. One or more operations of the method may be performed by embodiments disclosed herein, e.g., by a computer such as the computer system 22 of FIG. 1. FIG. 5 is a flowchart of the example method, and FIGS. 6A to 6I show images 410 (410a to 410i) of an eye that has an IOL. For example, the eye image 410a shows the cornea 412 and the iris 414 of the eye.

The method starts at block 308, where a computer receives the image 410a of an eye that has an implanted IOL. The computer detects the iris plane 416 in an anterior volume, e.g., volume A 420, of the eye image 410a at block 310. The computer may first detect the iris 414 and/or the pupil in the image 410a to detect the iris plane 416. The anterior volume may represent a portion of the eye image that includes the anterior portion of the eye and may include, e.g., some, most, or all of the eye image 410a. As shown in the eye image 410b, the computer crops an iris sub-volume, e.g., the sub-volume B 422, around the iris plane 416 from the anterior volume at block 312. The iris sub-volume is used to generate an iris enface image, which is in turn used to locate the pupil. The iris sub-volume may include most of or all (e.g., 70 to 80, 80 to 90, and/or 90 to 100 percent) of the iris 414.

The computer creates an iris enface image, e.g., the enface image Bz 410c, from the iris sub-volume, at block 314. The iris enface image may be created from any suitable portion of the iris sub-volume, e.g., at an xy-plane within the iris sub-volume such as approximately (within, e.g., +2, 3, 4, or 5 millimeters of) at the middle of the iris sub-volume. At block 316, the computer detects the pupil 424 in the iris enface image. The pupil 424 may be detected by performing image processing that detects, e.g., a darker, circular shape in the iris enface image.

As shown in the eye image 410d, the computer crops a pupil sub-volume, e.g., the sub-volume C 426, relative to the iris plane 416 and the pupil 424 from the anterior volume at block 320. The pupil sub-volume is used to target the search space to where the IOL may be located. Accordingly, the computer may crop the pupil sub-volume generally posterior to the iris plane 416, e.g., posterior to the pupil portion of the iris plane 416. For example, most (e.g., 70 to 80, 80 to 90, and/or 90 to 100 percent) of the pupil sub-volume may be posterior to the iris plane 416 in the z-direction to approximate the average or expected location of the IOL. (An average IOL may have an anterior surface located at, e.g., 4 to 5 millimeters in the z-direction and a posterior surface located at, e.g., 5 to 6 millimeters in the z-direction.) The pupil sub-volume may have any suitable size, e.g., an xy-dimension that is approximately (within, e.g., +2, 3, 4, or 5 millimeters of) the diameter of the average or expected IOL and a z-dimension that is approximately (within, e.g., +2, 3, 4, or 5 millimeters of) the z-dimension of the average or expected IOL.

The computer creates a pupil enface image, e.g., the enface image Cz 410e, from the pupil sub-volume at block 322. The pupil enface image is used to locate Purkinje images. The pupil enface image may be created from any suitable portion of the pupil sub-volume. For example, the pupil enface image may be created at an xy-plane within the pupil sub-volume that approximates (within, e.g., +2, 3, 4, or 5 millimeters of) the average and/or expected z-location of the IOL. The computer detects the brightest spots of the pupil enface image as Purkinje P3 and P4 image candidates at block 324. For example, the computer performs image processing to detect lighter areas of the image.

The computer determines the xy-location P3_xy of the P3 image and the xy-location P4_xy of the P4 image in the pupil enface image at block 325. The computer detects the z-location P3_z of the P3 image and the z-location P4_z of the P4 image according to the maximum intensities of the P3 image and the P4 image, respectively, at block 326. The xy-location P3_xy and the z-location P3_z of the P3 image form the xyz-location P3_xyz of the P3 image, that is, the P3 point that yields the P3 image. Similarly, the xy-location P4_xy and the z-location P4_z of the P4 image form the xyz-location P4_xyz of the P4 image, that is, the P4 point. In the example, the graph 410f shows the intensity of the P3 image (labeled I_P3_xyz) and the intensity of the P4 image (labeled I_P4_xyz). The maximum intensity I_P3_xyz of P3 is at approximately z=50 and the maximum intensity I_P4_xyz of P4 is at approximately z=275.

In general, the posterior side of the IOL yields the P4 image, and the anterior side yields the P3 image, so the z value of the P4 image is typically greater than the z value of the P3 image. However, if P3_z>P4_z at block 330, then the computer sets P4_xyz=P3_xyz at block 332. Otherwise, the method proceeds to block 338.

As shown in the image 410g, the computer crops a posterior volume, e.g., sub-volume D 428, around the point P4_xyz from the pupil sub-volume at block 328. The posterior volume is used to target the search space of the location of the posterior surface of the IOL. The posterior volume may have any suitable length in the z-direction (e.g., 0.5 to 2, 2 to 4, 4 to 6, and/or 6 to 8 millimeters) and any suitable area in the xy-direction (e.g., within +2 millimeters of the area of the pupil sub-volume in the xy-direction). As shown in image 410h, the computer performs segmentation of the posterior IOL surface 432 (S_backside_IOL) in the posterior volume with the point P4_xyz as a forced point on the surface 432 at block 340. For example, the computer may perform image processing to detect lighter areas indicating the posterior IOL surface 432, e.g., lighter areas with the point P4_xyz.

As shown in the image 410i, the computer crops an anterior sub-volume, e.g., sub-volume E 430, which is anterior to the posterior IOL surface 432, from the pupil sub-volume, at block 342. The anterior volume is used to target the search space for the location of the anterior surface of the IOL. The anterior volume may have any suitable dimensions (e.g., in the ranges as described herein for the posterior surface). At block 344, the computer performs segmentation of the anterior IOL surface 434 (S_frontside_IOL) in the anterior sub-volume. For example, the computer performs image processing to detect lighter areas indicating the anterior IOL surface 434, with the point P3_xyz as a forced point of the surface 434. As another example, the computer estimates the location of the anterior IOL surface 434 using the location of the posterior IOL surface 432 and the expected shape of the IOL.

At block 346, the computer determines the location of the IOL according to the surfaces of the IOL. For example, the location of the IOL may be given as the location(s) of the anterior IOL surface and/or the posterior IOL surface. As another example, the location of the IOL may be given as the location(s) of the anterior IOL surface and/or the posterior IOL surface relative to another structure of the eye (e.g., the iris plane and/or the cornea). The computer may then output the location of the IOL to a display.

A component (such as the computer system 22) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112 (f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112 (f).

What is claimed:

1. A system for detecting an intraocular lens (IOL) in an image of an eye, comprising:
an imaging system configured to generate the image of the eye, the eye having a plurality of structures, the plurality of structures comprising the IOL; and
a computer system configured to:
receive the image of the eye from the imaging system;
detect one or more Purkinje images in the image of the eye, a Purkinje image of the one or more Purkinje images comprising a reflection from a structure of the plurality of structures of the eye;
use the one or more Purkinje images to detect one or more surfaces of the IOL; and
determine a location of the IOL according to the one or more surfaces of the IOL.

2. The system of claim 1, the imaging system comprising an optical coherence tomography (OCT) imaging system configured to generate the image of the eye as an OCT image.

3. The system of claim 1, the computer system further configured to detect the one or more Purkinje images in the image of the eye by:
detecting an iris and a pupil in the image of the eye;
creating one or more enface images according to the iris and the pupil; and
detecting the one or more Purkinje images in at least one enface image of the one or more enface images.

4. The system of claim 1, the computer system further configured to detect the one or more Purkinje images in the image of the eye by:
detecting an iris plane in the image of the eye;
creating an iris enface image using the iris plane;
detecting a pupil in the iris enface image;
creating a pupil enface image posterior to the iris plane; and
detecting the one or more Purkinje images in the pupil enface image.

5. The system of claim 1, the computer system further configured to use the one or more Purkinje images to detect the one or more surfaces of the IOL by:
defining a search volume according to a Purkinje image of the one or more Purkinje images; and
searching the search volume for a surface of the one or more surfaces of the IOL.

6. The system of claim 1, the computer system further configured to use the one or more Purkinje images to detect the one or more surfaces of the IOL by:
determining an enface location of a point associated with a Purkinje image of the one or more Purkinje images, the point corresponding to a Purkinje point reflecting light that yields the Purkinje image; and
detecting a surface of the one or more surfaces of the IOL according to the enface location of the point associated with the Purkinje image.

7. The system of claim 1, the computer system further configured to use the one or more Purkinje images to detect the one or more surfaces of the IOL by:
determining a depth location of a point associated with a Purkinje image of the one or more Purkinje images, the point corresponding to a Purkinje point reflecting light that yields the Purkinje image; and
detecting a surface of the one or more surfaces of the IOL according to the depth location of the point associated with the Purkinje image.

8. The system of claim 1, the computer system further configured to use the one or more Purkinje images to detect the one or more surfaces of the IOL by:
determining a location of a point associated with a Purkinje image of the one or more Purkinje images, the point corresponding to a Purkinje point reflecting light that yields the Purkinje image; and
constraining a surface of the one or more surfaces of the IOL to include the location of the point.

9. The system of claim 1, the computer system further configured to use the one or more Purkinje images to detect the one or more surfaces of the IOL by:
detecting a posterior surface of the one or more surfaces of the IOL; and
searching a volume that is anterior to the posterior surface for an anterior surface of the one or more surfaces of the IOL.

10. The system of claim 1, the computer system further configured to use the one or more Purkinje images to detect the one or more surfaces of the IOL by:
detecting a posterior surface of the one or more surfaces of the IOL; and
detecting an anterior surface of the one or more surfaces of the IOL according to the posterior surface and a shape of the IOL.

11. The system of claim 1, the computer system further configured to:
detect the one or more Purkinje images in the image of the eye by:
detecting a fourth Purkinje (P4) image of the one or more Purkinje images, the P4 image reflected from a posterior surface of the one or more surfaces of the IOL; and
use the one or more Purkinje images to detect the one or more surfaces of the IOL by:
using the P4 image to detect the posterior surface of the one or more surfaces of the IOL.

12. The system of claim 1, the computer system further configured to:
detect the one or more Purkinje images in the image of the eye by:
detecting a fourth Purkinje (P4) image of the one or more Purkinje images, the P4 image reflected from a posterior surface of the one or more surfaces of the IOL; and
use the one or more Purkinje images to detect the one or more surfaces of the IOL by:
determining a location of a P4 point that yields the P4 image;
defining a posterior search volume according to the location of the P4 point; and
searching the posterior search volume for the posterior surface of the one or more surfaces of the IOL.

13. The system of claim 1, the computer system further configured to:
detect the one or more Purkinje images in the image of the eye by:
detecting a fourth Purkinje (P4) image of the one or more Purkinje images, the P4 image reflected from a posterior surface of the one or more surfaces of the IOL; and
use the one or more Purkinje images to detect the one or more surfaces of the IOL by:
determining a location of a P4 point that yields the P4 image; and
constraining a surface of the one or more surfaces of the IOL to include the location of the P4 point.

14. The system of claim 1, the computer system further configured to:
detect the one or more Purkinje images in the image of the eye by:
detecting a fourth Purkinje (P4) image of the one or more Purkinje images, the P4 image reflected from a posterior surface of the one or more surfaces of the IOL; and
use the one or more Purkinje images to detect the one or more surfaces of the IOL by:
determining a depth of a maximum intensity of the P4 image; and
detecting the posterior surface using the depth of the maximum intensity of the P4 image.

15. The system of claim 1, the computer system further configured to:
detect the one or more Purkinje images in the image of the eye by:
detecting a third Purkinje (P3) image of the one or more Purkinje images, the P3 image reflected from an anterior surface of the one or more surfaces of the IOL; and
use the one or more Purkinje images to detect the one or more surfaces of the IOL by:
using the P3 image to detect the anterior surface of the one or more surfaces of the IOL.

16. The system of claim 1, the computer system further configured to:
detect the one or more Purkinje images in the image of the eye by:
detecting a third Purkinje (P3) image of the one or more Purkinje images, the P3 image reflected from an anterior surface of the one or more surfaces of the IOL; and
use the one or more Purkinje images to detect the one or more surfaces of the IOL by:
determining a location of a P3 point that yields the P3 image;
defining an anterior search volume according to the location of the P3 point; and
searching the anterior search volume for the anterior surface of the one or more surfaces of the IOL.

17. The system of claim 1, the computer system further configured to:
detect the one or more Purkinje images in the image of the eye by:
detecting a third Purkinje (P3) image of the one or more Purkinje images, the P3 image reflected from an anterior surface of the one or more surfaces of the IOL; and
use the one or more Purkinje images to detect the one or more surfaces of the IOL by:
determining a location of a P3 point that yields the P3 image; and
constraining a surface of the one or more surfaces of the IOL to include the location of the P3 point.

18. The system of claim 1, the computer system further configured to:
detect the one or more Purkinje images in the image of the eye by:
detecting a third Purkinje (P3) image of the one or more Purkinje images, the P3 image reflected from an anterior surface of the one or more surfaces of the IOL; and
use the one or more Purkinje images to detect the one or more surfaces of the IOL by:
determining a depth of a maximum intensity of the P3 image; and
detecting the anterior surface using the depth of the maximum intensity of the P3 image.

19. The system of claim 1, the computer system further configured to:
display the image of the eye;
receive a user selection of a search volume; and
search the search volume for the one or more surfaces of the IOL.

20. The system of claim 1, the computer system further configured to:
display the image of the eye;
display a search volume on the image of the eye; and
search the search volume for the one or more surfaces of the IOL.
